(19) United States
(12) Patent Application Publication
(10) Pub. No.: US 2013/0230566 A1
(43) Pub. Date: Sep. 5, 2013

(54) WOUND HEALING COMPOSITIONS AND ASSOCIATED METHODS

(71) Applicants: Cary Jake Lambert, Jr., Winter Haven, FL (US); Thomas Slaughter, Lakeland, FL (US)

(72) Inventors: Cary Jake Lambert, Jr., Winter Haven, FL (US); Thomas Slaughter, Lakeland, FL (US)

(21) Appl. No.: 13/858,330

(22) Filed: Apr. 8, 2013

Related U.S. Application Data
(62) Division of application No. 13/184,059, filed on Jul. 15, 2011.

(51) Int. Cl. A61L 24/04

(52) U.S. Cl. CPC A61L 24/046
USPC 424/400; 514/772.1

(57) ABSTRACT

In one embodiment, the present disclosure provides a composition including an aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, and a substrate. In another embodiment, the present disclosure provides a method of attaching a substrate to a tissue by applying a substrate to a tissue and applying an aqueous solution comprising a poloxamer to the tissue in an amount sufficient for the poloxamer to hold the substrate to the tissue. In a third embodiment, the present disclosure a method of facilitating wound closure by applying an original substrate to a wound, applying an original aqueous solution comprising a poloxamer to the wound in an amount sufficient for the poloxamer to hold the substrate to the wound, and maintaining a substrate and poloxamer on the wound until wound closure.

### RELATED APPLICATIONS

This application is a divisional of U.S. patent application Ser. No. 13/184,059 filed Jul. 15, 2011, the contents of which is hereby incorporated by reference in its entirety.

### BACKGROUND

Soft tissue defects or open wounds may result from a variety of events including, but not limited to, trauma, burns, diabetic ulcers, severe infections, such as necrotizing fasciitis, venous stasis disease, and pressure ulcerations. The resultant wounds may present a formidable clinical challenge due the variability of depth, size, and location as well as other issues such as the resultant exposure of bone, fascia, muscle, or cartilage. In addition, wounds may be further influenced by individual patient indications, such as poor nutrition, diabetes, local and/or systemic infections, renal failure, steroid use, venous insufficiency, peripheral artery disease, congestive heart failure, low cardiac output, and shock states. Furthermore, wounds often go through distinct phases of wound healing, which generate very different cells and tissue responses. These different cells and tissue responses may contribute to variability in wound contraction, scar formation, and capillary in-growth.

Biologic and biodegradable substrates (such as grafts and meshes) are commonly used to facilitate wound closure. Examples of biologic and biodegradable substrate products available on the market include Apligraf® (Organogenesis, Inc., Canton, Mass.), Alloderm® (LifeCell Corporation, Branchburg, N.J.), Oasis® (Healthpoint Biotherapeutics, Fort Worth, Tex.), Graft Jacket® (Wright Medical Technology, Inc., Arlington, Tenn.), Dermagraft® (Advanced Biohealing, Inc., Westport, Conn.), Integra™ (Integra Life Sciences Corp., Plainsboro, N.J.), Cymetra® (LifeCell Corporation, Branchburg, N.J.), Transcyte® (Advanced Biohealing, Inc., Westport, Conn.), E-Z-Derm® (Brennen Medical, LLC, St. Paul, Minn.), Orcel® (Forticell Bioscience, Inc. New York, N.Y.). Collagen is one of the most common biologic graft products used in open wounds. Collagen may be derived from autologous skin, porcine dermis, porcine small intestine sub-mucosa, bovine dermis, bovine pericardium, cadaver skin, and umbilical cells. Examples of collagen containing substrates include Biopad® (Euroresearch, S.R.L., Milan, Italy), Fibracol® (Johnson & Johnson, New Brunswick, N.J.), Purocol® (Medline Industries, Inc., Mundelein, Ill.), Helitape® (Lutipold Pharmaceuticals, Shirley, N.Y.), Promogran® (Systagenix, Gatwick, UK) and Promogran Silver® (Systagenix, Gatwick, UK). Other products may include biodegradable polymers such as polymer nanofiber mesh (PNF), hyaluronic acid (hyaluranon), polyesters, polyglycolides, polylactides, polyorthoesters, polyanhydrides, polyphosphozenes, and polyurethane. Examples of grafts containing biodegradable polymers include Polymem® (Ferris Manufacturing Corp., Burr Ridge, Ill.), Polymem Silver® (Ferris Manufacturing Corp., Burr Ridge, Ill.), and BIO-A® (W. L. Gore & Associates, Inc, Flagstaff, Ariz.). These polymers may be tailored to have various mechanical properties and also different degradation kinetics. Synthetic polymers can also be fabricated into many different shapes with pore morphologic features that are conducive to tissue in-growth.

Biologic and biodegradable tissue, grafts, and meshes may be attached and adhered to wounds in a variety of ways. Methods include suturing with using sterile tape strips and simply wrapping the wound securely with materials such as Kerlex™ (Covidien, Mansfield, Mass.), Kling (Johnson & Johnson, New Brunswick, N.J.), or Coban® (3M, St. Paul, Minn.). Most biologic materials require moisture to remain viable. Antibiotics and other ointments may be applied to assist these grafts. Film-cement has also been used to keep grafts and meshes in place. Recently the use of negative pressure therapy, such as would vacuum therapies, has been shown to be effective in keeping such grafts in place, especially when there has been (exudative) fluid in these wounds.

Skin grafts and wounds have been shown to go through three distinct phases of healing including (1) plasmatic imbibition-creation of a fibrin layer which allows diffusion of nutrients by capillary action from the recipient bed, (2) inosculation-in which capillaries from recipient and donor align and establish a vascular network, and (3) neovascularization-in which there is actual in-growth of new vessels. This in-growth of new vessels is the key to successful incorporation of other materials into open wounds.

Despite the plethora of available grafting materials, wounds continue to heal slowly and take a significant amount of time to close. Failure to close and inefficient incorporation/in-growth of these grafts and meshes result from the many variables described above. The ability to bioengineer materials to be more conducive to incorporation into human tissue should improve healing tissue strength and decrease healing times while simultaneously decreasing the pain, disability, and suffering that patients have to endure. Improved healing of wounds should also decrease the cost of patient wound care.

One significant issue that remains to be resolved is the problem of attaching mesh, bio-products, and biodegradable graft materials to tissue and wound sites.

Adhering something as simple as collagen to a wound or ulcer is problematic and is it even more difficult with the various meshes and products available for use.

### SUMMARY

The present disclosure generally relates to wound healing. More particularly, the present disclosure relates to wound healing compositions including poloxamers and associated methods.

In one embodiment, the present disclosure provides a composition including an aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, and a substrate.

In another embodiment, the present disclosure provides a method of attaching a substrate to a tissue by applying a substrate to a tissue and applying an aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, to the tissue in an amount sufficient for the poloxamer to hold the substrate to the tissue.

In a third embodiment, the present disclosure provides a method of facilitating wound closure by applying an original substrate to a wound, applying an original aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, to the wound in an amount sufficient for the poloxamer to hold the substrate to the wound, and maintaining a substrate and poloxamer on the wound until wound closure.

Unless otherwise noted herein, concentration percentages are w/v.

The features and advantages of the present invention will be apparent to those skilled in the art. While numerous changes may be made by those skilled in the art, such changes are within the spirit of the invention.

### BRIEF DESCRIPTION OF THE DRAWINGS

Some specific example embodiments of the disclosure may be understood by referring, in part, to the following description and the accompanying drawings. The file of the present patent contains at least one drawing executed in color as determined by the U.S. Pat. and Trademark Office. Copies of this patent with color drawing(s) will be provided by the Patent and Trademark Office upon request and payment of the necessary fees.

FIG. 1A shows a wound before treatment. FIG. 1B shows a wound immediately after application of a pharmaceutical composition including a poloxamer and a substrate.

FIG. 2A shows another wound before treatment (A). FIG. 2B shows the wound at a later time after application of a pharmaceutical composition including a poloxamer and a substrate. FIG. 2C shows the wound at a still later time after application of a pharmaceutical composition including a poloxamer and a substrate. FIG. 2D shows the wound after wound closure.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DETAILED DESCRIPTION

The present disclosure generally relates to wound healing. More particularly, the present disclosure relates to wound healing pharmaceutical compositions including poloxamers and associated methods of their formation and use, including their use with substrates.

Certain embodiments discussed below describe a thermoreversible thermoplastic pharmaceutical composition including a poloxamer which is biologically stable yet also thermoreversible, and which may be used to adhere meshes, biomaterials, and substrates to tissues and larger structures.

One of the many advantages of the compositions described herein is that they may permit the adherence of varieties of meshes and graft materials to open tissue Other potential advantages of the compositions described herein is that they may provide superior sealing of wounds so as to decrease tissue fluid loss, they may allow for the suspension of a wide variety of substances to enhance/improve wound healing, they may be modified for each phase of wound healing, they may provide substances for treating underlying medical conditions such as insulin for diabetics or antibiotics for infections, they are not unreasonably expensive, and they may be stored in liquid form at cooler temperatures while being applied as solid gel at room temperature, which allows for prolonged activity of additives, and they may allow for the creation of materials that can protect, heal, and close open wounds.

In one embodiment, the present disclosure provides pharmaceutical composition including a poloxamer. Poloxamers are a class of nonionic polyoxyethylene-polyoxypropylene block co-polymers with the general formula HO(C₂H₄O)ₐ(C₃H₆O)_{b}(C₂H₄O)ₐH, wherein a ranges from 12-101 and b from 20-56. At low concentrations (10⁻⁴ to 10⁻⁵%) in aqueous media they form monomolecular micelles, but at higher concentrations in aqueous media, they form mulitmolecular aggregates with a hydrophobic core and hydrophilic polyoxyethylene chains facing the aqueous medium

In numerous studies with Pseudomonas aeruginosa, poloxamers have been shown to decrease the bacterial adherence to surfaces up to 94%. Furthermore, studies with staphylococci show that poloxamer 407 inhibits adherences from 77-99.9% and also show that residual bacteria are more susceptible to antibiotic activity. In addition to the in-adhesive effects on bacteria, poloxamers also have mucoadhesive properties. The mucoadhesive properties may allow for the attachment of the gel to the biological site of action. With the addition of preservatives or other antimicrobial substances, these properties may allow a gel to maintain an aseptic environment for long periods of time, reducing and perhaps eliminating the number and frequency of dressing changes. This may be especially useful when used internally, prior to suturing.

In certain embodiments, the pharmaceutical composition may include at least one poloxamer selected from the group consisting of Poloxamers L61, L64, 101, 105, 108, 122, 123, 124, 181, 182, 183, 184, 185, 188, 212, 215, 217, 231, 234, 235, 237, 238, 282, 284, 288k, 331, 333, 334, 335, 338, 401, 402, 403, 407, 105 Benzoate, and 182 Dibenzoate. In certain preferred embodiments, the pharmaceutical composition may include Poloxamer 407. Poloxamer 407 is a triblock copolymer with the general formula of E₁₀₆P₇₀E₁₀₆ with an average molecular mass of 13000 daltons. Poloxamer 407 consists of a central hydrophobic block of propylene glycol flanked by two hydrophilic blocks of polyethylene glycol. The approximate length of the two PEG blocks is 101 repeat units while the approximate length of the propylene glycol block is 56 repeat units.

The pharmaceutical compositions may include any amount of poloxamer. In certain embodiments, the pharmaceutical composition may include an amount of poloxamer sufficient to form a poloxamer gel. In certain embodiments, the pharmaceutical composition may have a poloxamer concentration in an amount in the range of from about 1% to about 50% weight/volume of the pharmaceutical composition. In certain embodiments, the pharmaceutical composition may have a poloxamer concentration in an amount in the range of from about 1% to about 35% by weight/volume of the pharmaceutical composition. In certain embodiments, the pharmaceutical composition may have a poloxamer concentration in an amount in the range of from about 20% to about 35% weight/volume of the pharmaceutical composition. In certain embodiments, the pharmaceutical composition may have a poloxamer concentration of about 30% weight/volume of the pharmaceutical composition.

In certain embodiments, the pharmaceutical composition may include a dispersion medium. Any type of dispersion medium which may hydrate a poloxamer to form a poloxamer gel may be used. In particular examples, the dispersion media may be aqueous. Examples of suitable dispersion media include water, sterile water, saline solutions, dextrose solutions, lactated solutions, ribose or other sugars solutions, and combinations thereof. In certain embodiments, the dispersion media may be present in the pharmaceutical composition in an amount sufficient to form a poloxamer gel. In certain embodiments, the pharmaceutical composition may have a dispersion media concentration in an amount in the range of from about 50% to about 99% by weight/volume of the pharmaceutical composition. In certain embodiments, the pharmaceutical composition may have a dispersion media concentration in an amount in the range of from about 65% to about 99% by weight/volume of the pharmaceutical composition. In certain embodiments, the pharmaceutical composition may have a dispersion media concentration in an amount in the range of from about 65% to about 80% by weight/volume of the pharmaceutical composition. In certain embodiments, the pharmaceutical composition may have a poloxamer concentration of about 70% by weight/volume of the pharmaceutical composition.

In certain embodiments, the pharmaceutical composition may be capable of forming a poloxamer gel. At low temperatures in aqueous solutions, hydration layers may surround the poloxamer molecules, resulting in a micelle formation. At higher temperatures, including body temperatures, hydrophobic associations may be favored and may result in the formation of a phase containing hexagonal-packed cylinders, causing a gelation of the poloxamer. Reverse thermal gelation may occur if the temperature is again lowered, which may cause the poloxamer to revert back to its micelle form. This thermo-reversible physical property allows for the coating a wound, cavity, joint, or organ with a highly amorphous liquid that will turn viscous and more solid-like at higher temperature. Internal formation of the hexagonal cylinders may provide support structures for an exogenous material to be placed into the poloxamer at the site of application. For example, at concentrations above 16% w/v, many poloxamer gels are in a micelle formation at 5-10° C. At 15-20° C. these gels form the packed cylinder configuration.

The viscosity of the poloxamer gel depends on both the temperature and the concentration of the poloxamer. Dilute aqueous solutions display Newtonian flow, while at concentrations above 10% the poloxamer solution begins to display plastic flow. The sol-gel transition temperature of aqueous solutions of poloxamer 407 ranges from 15-25° C. at poloxamer concentrations exceeding 16%. The addition of other agents, such as salts that have multivalent anions, may reduce the ability of a poloxamer solution to fully form a gel.

The physical nature of poloxamers in the pharmaceutical composition (hydrophobic core with hydrophilic chains facing the medium), may further allow hydrogen bonding, Van der Waals forces, and local forces to play a role in adhering substrates or other materials to the application site on or in the body. Due to the nature of the pharmaceutical compositions, in certain embodiments, macromolecular structures may be held and bonded onto the application site, while still allowing movement of ancillary products by diffusion processes and physical in-growth of tissue through the sol-gel matrix. Furthermore, micro-molecular forces may have adhesive effects on local flora.

In general, poloxamer gels of the current disclosure may further be useful in allowing the formation of a solution or suspension of materials that will not readily form a solution or suspension in water. For instance, Nifedipine may form a suspension in a poloxamer gel.

In certain embodiments, the pharmaceutical composition may further include one or more additives. In certain embodiments, the aqueous nature of the dispersion media allows for hydrophilic additives to readily dissolve and move from the poloxamer gel to either a substrate or to a wound or biological site, or both via simple diffusion and first order kinetics. Molecules that are normally not soluble in an aqueous environment may be incorporated into the gel by suspension in the physical lattice of the gel form or by incorporation into the hydrophobic regions of the micelle arrangement. These molecules and substances may then be pushed through the gel by nature of the hexagonal packed cylinders, allowing the gel to function as a depot for even non-dissolvable particles. In general, unless otherwise specified, the following additives may be present in the pharmaceutical composition at concentrations between 0.001-50% (w/v).

The pharmaceutical composition may contain additives to prevent infection (antibiotics) and minimize blood loss (vasoconstrictors) or hemostatic agents, or a topical numbing agent (topical anesthetics).

In certain embodiments, the pharmaceutical composition may include a cellulose additive. Examples of suitable cellulose additives include microcrystalline cellulose, cellulose derivatives, ethyl cellulose, methyl cellulose, carboxymethyl cellulose, hydroxypropylmethyl cellulose, hydroxypropyl cellulose, gums, acacia gum, tragacanth gum, carrageenans, pectine, carbomers, modified starch, colloidal aluminum silicates, and combinations thereof.

In certain embodiments, the pharmaceutical composition may further include one or more preservatives. Suitable preservatives may include benzalkonium chloride, ethanol, carbopol, polycarbophil, propylene glycol, ethylene glycol, sodium chloride, sodium alginate, fatty acids, lipids, hydrophobic substances, parabens, alcohols, ethyl alcohol, benzyl alcohol, potassium sorbate, creosol, terpene derivatives, and combinations thereof.

In certain embodiments, the pharmaceutical composition may further include one or more pH adjusting agents. Examples of suitable pH adjusting agents include sodium hydroxide, hydrochloric acid, sodium bicarbonate, and combinations thereof. In general, formation of packed cylinders is more dependant on temperature on poloxamer concentration than on pH. pH adjustments may facilitate inclusion or stability of additives, such as antibiotics. Some, pH adjusting agents, such a hydrocholoric acid, may be used sparingly due to negative effects on gel strength and adhesion strength.

In certain embodiments, the pharmaceutical composition may further include one or more clotting agents. Examples of suitable clotting agents include fibrinogen clotting factors, kaolin, fibrillar avitene, DDAVP, amicar, protamine, and combinations thereof.

In certain embodiments, the pharmaceutical composition may further include one or more compounds and entities known to be anti-oxidative, oxidative, or reductive in nature. Example material include antioxidants such as ascorbic acid, Vitamin e, and selenium, oxidative materials such as potassium permanganate, sodium hypochlorite, and ozone, and reductive materials such as sulfur dioxides.

In certain embodiments, the pharmaceutical composition may further include one or more compounds and entities known to treat or mitigate aerobic or anaerobic conditions or conditions including, but not limited to, oxidative stress or high carbon-monoxide. Suitable compounds may include methylene blue or other monoamine oxidase inhibitors.

In certain embodiments, the pharmaceutical composition may further include one or more enzymes or entities that are free-radical scavengers. Suitable examples include ascorbic acid, glutathione, DMPS, DMSA, NAC, methionine, and superoxide dismutase.

In certain embodiments, the pharmaceutical composition may further include one or more compounds that prevent or treat keloid or hypertrophic type scars. Suitable examples include silicone, cyclopentasiloxane, phenyl trimethicone, interferon (alpha, beta, and gamma), imiquimod, tacrolimus, chochicine, betacarotine, and ace inhibitors.

In certain embodiments, the pharmaceutical composition may further include one or more compounds that can remove or reduce the visibility of tattoos. Suitable examples include phytolacca, aloe vera sap, calcium flouride, graphites, alpha hydroxy acid, beta hydroxy acid, tricholoracetic acid, and phenols.

In certain embodiments, the pharmaceutical composition may further include one or more compounds that can treat psoriasis. Suitable examples include tazorac, retinoids, coal tar, anthralin, kerotolytics, acitretin, and calcineunin.

In certain embodiments, the pharmaceutical composition may further include one or more concentrated gasses. Suitable examples include oxygen, nitrogen, and carbon dioxiate. The concentrations of these gases may differ depending on the nature of the substrate and the location of treatment, stage of wound, or other modality.

In certain embodiments, the pharmaceutical composition may further include one or more pigments or dyes. The pigments or dies may provide for better visualization, protection, cosmetic, or to help either the local environment or facilitate the substrate.

In certain embodiments, the pharmaceutical composition may further include one or more aromatic ingredients, deodorants, or masking agents. Suitable examples include fixed oils and aromatic oils such as spearmint, peppermint, and/or eucalyptus, as well as cinnamon and tea tree oils. May also include, but is not limited to fragrances such as liliac, baby powder, or other synthetic or botanical oils, resins, extracts, or derivatives thereof.

In certain embodiments, the pharmaceutical composition may further include one or more antineoplastic agents.

In certain embodiments, the pharmaceutical composition may further include one or more enzymatic debridement agents including but not limited to metalloproteinases, collagenases, sultilains ointment, trypsin, chymotrypsin, deoxyribonuclease, and papain urea.

In certain embodiments, the pharmaceutical composition may further include one or more substances added for stability and maintain a specific viscosity at different temperatures, which include but are not limited to temperatures needed for storage and physiologic temperatures. For instance, ethanol may be added to increase the temperature at which the material transforms from a micelle-based structure to packed cylinders, thereby allowing the poloxamer gel to remain fluid at higher temperatures. Carbopol may be used to decrease the temperature at which packed cylinder formation occurs and may also increase viscosity and adhesion of the pharmaceutical material.

In certain embodiments, the pharmaceutical composition may further include one or more anesthetic drugs. The anesthetic drugs may be local or systemic anesthetics. Examples of suitable anesthetic drugs include cocaine, procaine, amethocaine, chloroprocaine, lignocaine, prilocaine, mepivacaine, and bupivacaine. The systemic anesthetic agents may include all narcotic and non-narcotic agents.

In certain embodiments, the pharmaceutical composition may further include one or more naturally-occurring compounds, herbs, extracts, or byproducts, derived in part or in whole, from animal or plant tissues. Examples include, but are not limited to: shark cartridge, coral calcium, oak extract (quercus robur), gensing, turmeric, anise oil, rosemary oil, Siberian gensing, CoQ10, glucosamine, melanin, and melatonin.

In certain embodiments, the pharmaceutical composition may further include one or more endothelial growth factors, insulin, insulin receptor modulators, fructose diphosphate, extracellular matrix, vitamins, trace elements, or cofactors, amino acids, peptides, proteins and enzymes, as well as intracellular and extracellular proteins and hormonal factors, growth hormones, and stem cells.

In certain embodiments, the pharmaceutical composition may further include one or more pharmaceuticals to enhance the action and prevent the breakdown of biomaterial substrates from natural or artificial sources such as pathogenic bacterium. Examples of these materials may include antimicrobials such as macrolides, quinolones, beta-lactams and beta-lactam containing materials, phenols, sulfur-containing, vancomycin, penicillins, cyclic lepopeptides, rifampicin, silver-based compounds, rifamycin, azole antifungals, terbenifiine, amphotericin, undecylenic acid, and other antifungal, antibacterial, and antiprotozoals.

In some embodiments, poloxamer compositions may be applied alone to a wound. In particular embodiments it may be applied to an internal wound, such as a surgical wound, or a deep wound. In these and other embodiments, application of poloxamer compositions between tissues may adhere the tissues to one another and thereby facilitate healing. For instance, it may be used in wound closure. Such products may specifically include antibiotics or growth factors. Poloxamer compositions used in these situations may provide additional benefits, such as prevention of infection or abscess. Infection and abscess prevention properties may be particularly useful in contaminated wounds.

In certain embodiments, the present disclosure provides for a composition including the pharmaceutical composition and meshes, biomaterials, or substrates (referred to collectively as “substrates” herein), including wound healing substrates. In certain embodiments, the pharmaceutical composition may be impregnated into a substrate to enhance adherence onto tissue and open wounds. In certain embodiments, this composition may be fashioned into a gauze-like, wrap, or cling type product for the purpose of adhering, protecting, or hastening wound closure. In certain embodiments, the substrate may be biologic, biodegradable, and/or bioadsorbable. In certain embodiments, for example when the substrate is a collagen wound dressing such as a BioPad® Biopad® (Euroresearch, S.R.L., Milan, Italy), a poloxamer gel may be applied to the edges and top of the substrate, and if desired, covered with non-adherent or standard wound dressing.

In certain embodiments, the substrate may include a biodegradable and/or bioadsorbable polymer. Examples of suitable biodegradable polymers include polymer nanofiber mesh (PNF), hyaluronic acid (hyaluranon), polyesters, polyglycolides, polylactides, polyorthoesters, polyanhydrides, polyphosphozenes, polyurethanes, and polyvinyl alcohols.

In particular embodiments, the substrate may include a material to be implanted inside the body, such as polytetrafluorethylene or other non-biodegradable materials. For instance it may include a graft or other prosthetic material. In some such embodiments the poloxamer gel may be applied to such a substrate at the time of implantation in the body. This poloxamer gel may include antibiotics, anti-inflammatory medication, chemotherapeutic agents, growth factors, stem cells, extracellular matrix, or clotting factors.

In certain embodiments, the substrate may include autologous tissue. The substrate may be cultivated tissue collected from living human or animal donors or cadavers including those grown from stem cells, or have characteristics of human tissue, including dermis-derived tissues and other epithelial sources including but not limited to umbilical cells or small intestinal mucosa.

In certain embodiments, the substrate may include a vascular graft, or graft from any tissue, organ, or biological system.

In certain embodiments, the substrate may include an artificial material. Examples of suitable artificial material include synthetic meshes, screens, and acrylates.

In certain embodiments, the substrate may include a more complex materials including synthetic vales, synthetic joints, bone matrices, as well as synthetic, cryopreserved, or cadaveric vascular grafts.

In certain embodiments, the substrate may include a biologic or biodegradable substrate product available on the market.

In one example, the substrate may include a skin-like material, such as Apligraf® (Organogenesis, Inc., Canton, Mass.). Like human skin, Apligraf® consists of living cells and structural proteins. The lower dermal layer combines bovine type 1 collagen and human fibroblasts (dermal cells), which produce additional matrix proteins. The upper epidermal layer is formed by promoting human keratinocytes (epidermal cells) first to multiply and then to differentiate to replicate the architecture of the human epidermis.

Dermagraft® (Advanced Biohealing, Inc., Westport, Conn.) is another skin-like material suitable for use as a substrate. Dermagraft® is a cryopreserved human fibroblast-derived dermal substitute. It is composed of fibroblasts, extracellular matrix, and a bioabsorbable scaffold. Dermagraft® is manufactured from human fibroblast cells derived from newborn foreskin tissue. During the manufacturing process, the human fibroblasts are seeded onto a bioabsorbable polyglactin mesh scaffold. The fibroblasts proliferate to fill the interstices of this scaffold and secrete human dermal collagen, matrix proteins, growth factors, and cytokines to create a three-dimensional human dermal substitute containing metabolically active living cells. Dermagraft® does not contain macrophages, lymphocytes, blood vessels, or hair follicles.

Transcyte® (Advanced Biohealing, Inc., Westport, Conn.), another suitable substrate, is a human fibroblast-derived temporary skin substitute consisting of a polymer membrane and neonatal human fibroblast cells cultured under aseptic conditions in vitro on a nylon mesh. Prior to cell growth, this nylon mesh is coated with porcine dermal collagen and bonded to a polymer membrane (silicone). This membrane provides a transparent synthetic epidermis when applied. As fibroblasts proliferate within the nylon mesh, they secrete human dermal collagen, matrix proteins and growth factors.

Orcel® (Forticell Bioscience, Inc. New York, N.Y.) forms another substrate usable in the present disclosure. Orcel® is a bilayered cellular matrix in which normal human allogeneic skin cells (epidermal keratinocytes and dermal fibroblasts) are cultured in two separate layers into a Type I bovine collagen sponge. Donor dermal fibroblasts are cultured on and within the porous sponge side of the collagen matrix while keratinocytes, from the same donor, are cultured on the coated, non-porous side of the collagen matrix.

In another example, the substrate may include a regenerative tissue matrix derived from living organisms. Alloderm® (LifeCell Corporation, Branchburg, N.J.) is one such material. AlloDerm® Tissue Matrix is produced through a unique non-damaging process that allows the body to mount its own tissue regeneration process. Donated human skin tissue is aseptically processed to remove the epidermis and cells that can lead to tissue rejection and graft failure. The result is an intact acellular matrix of natural biological components that promotes rapid revascularization, white cell migration and cell repopulation. AlloDerm® may also be in the form of Cymetra® Micronized AlloDerm® Tissue, which is a micronized particulate form of AlloDerm® Tissue Matrix. Like AlloDerm® Tissue Matrix, Cymetra® Micronized AlloDerm® Tissue contains collagens, elastin, proteins, and proteoglycans. The collagens and elastin provide structure for cell repopulation, while the proteoglycans and proteins allow the patient's own cells to initiate revascularization and cell repopulation. Cymetra® may be particularly suited for injection as a minimally invasive tissue graft.

Oasis® (Healthpoint Biotherapeutics, Fort Worth, Tex.) is another suitable tissue matrix material usable as a substrate. The Oasis® Wound Matrix is comprised of porcine-derived, acellular small intestine submucosa (SIS) material to form a matrix-based product compatible with human tissue.

Graft Jacket® (Wright Medical Technology, Inc., Arlington, Tenn.), forms another suitable substrate material. Graft Jacket® Matrix is made from donated human skin, which undergoes a process that removes the epidermis and dermal cells. This process allows the body to accept the matrix and reduces the rejection response. The processing steps that yield the Graft Jacket® Matrix sufficiently preserve the human dermal tissue, including its native protein, collagen structure, blood vessel channels and essential biochemical composition, to allow cellular repopulation and revascularization through the body's natural healing process.

E-Z-Derm™ (Brennen Medical, LLC, St. Paul, Minn.) is a suitable skin xenograft substrate material. E-Z-Derm™ is formed from frozen, irradiated porcine skin and contains a dermal and an epidermal layer.

In still another example, the substrate may be a complex wound dressing. For instance, it may be the Integra™ (Integra Life Sciences Corp., Plainsboro, N.J.) wound dressing. The Integra™ Bilayer Matrix Wound Dressing is a wound care device made of a porous matrix of cross-linked bovine tendon collagen and glycosaminoglycan and a semi-permeable polysiloxane (silicone layer). The semi-permeable silicone membrane controls water vapor loss, provides a flexible adherent covering for the wound surface and adds increased tear strength to the device. The collagen-glycosaminoglycan biodegradable matrix provides a scaffold for cellular invasion and capillary growth.

In a further example, the substrate may include a collagen wound dressing. Example materials include Biopad® (Euroresearch, S.R.L., Milan, Italy), which is constituted exclusively by lyophilized type 1 native heterologous collagen extracted from horse flexor tendon, and Helitape® (Lutipold Pharmaceuticals, Shirley, N.Y.), formed from pure bovine collagen.

Collagen wound dressing substrates may also include mixed collagen materials, such as Fibracol® (Johnson & Johnson, New Brunswick, N.J.), which also contains alginate, Purocol® (Medline Industries, Inc., Mundelein, Ill.), which contains pure native collagen and occasionally additives, such as silver, and Promogran® and Promogran Prisma® (Systagenix, Gatwick, UK), which also contain oxidized regenerated cellulose and, in the later product, silver.

In still another example, the substrate may include a graft containing a biodegradable polymer. Suitable materials include Polymem® and Polymem Silver® (Ferris Manufacturing Corp., Burr Ridge, Ill.), a complex foam-based material containing glycerin, a starch co-polymer, a polyurethane membrane, a semipermeable thin film backing, a surfactant cleansing agent and, in the later, silver. Other suitable graft substrate materials include BIO-A® (W. L. Gore & Associates, Inc, Flagstaff, Ariz.), a synthetic bioabsorbable scaffold.

A biodegradable material may be sterilized and used in conjunction or in place of the substrate. The substrate, either synthetic, collagen in origin, or biodegradable, may be in pieces or in strips or rolls, but will still be flexible enough to be able to pack into wounds.

In certain embodiments, the pharmaceutical composition may be sterilized. In another embodiment, it may be refrigeratable. Cooler temperatures may prolong the efficacy of the poloxamer and any additional substances.

The pharmaceutical composition of the disclosure may be packaged alone or with a substrate. In one embodiment, the substrate may be placed into a tray or packet of the poloxamer gel, with the gel coating the substrate, and excess poloxamer being present in and around the substrate to allow excess for adhering to the wound. In still a more specific embodiment, the whole tray or packet may be inverted or opened over a wound, with the tray or packet serving as another barrier to prevent infection, blood loss, or other deleterious effects as well as preserve the ability to adhere to the substrate. The packet may include synthetic mesh, collagen pad, or other bioabsorbable material. The package or tray may incorporate an acrylate or other adhesive strip to attach to an intact area of skin or clothing. The packaging can be colored or dyed to prevent observation if for the military, or of a highly reflective material (such as reflective Mylar for easy identification) for commercial uses.

With the addition of proper substrate and support materials or additives, embodiments of the present disclosure can facilitate a universal dressing and possibly universal graft incorporation, which may have applications in surgical wound closure and open wound healing. Furthermore, certain compositions discussed herein may be bioengineered to potentially blunt inflammatory response with vascular grafts, transplants, or prosthetic prosthesis.

In certain embodiments, the present disclosure provides a method of adhering a pharmaceutical composition to a wound, body surface, cavity, organ or other desired location in or on the body. The pharmaceutical composition may be applied as liquid for initial ease of use to an application site and then allowed to gel to a semisolid at body temperature. The pharmaceutical composition may then act as an additional substrate for tissue in-growth. The nature of the pharmaceutical compositions may further advance tissue re-growth, vessel in-growth, and wound repair by incorporating factors that enhance substrate effectiveness. In addition, the pharmaceutical composition may provide a way to deliver factors and substrates useful in healing to a wound site.

In certain embodiments, the pharmaceutical composition may be used to adhere biologic or biodegradable meshes, as well as skin (and other cellular) grafts to open wounds.

Furthermore, in certain embodiments, the pharmaceutical composition may simultaneously allow the introduction of growth structured exogenous materials, extracellular matrix, and other materials. These materials may be packaged in part or as a whole to form a universal graft or mesh enhancer. The pharmaceutical composition may be modified to provide a suitable environment for incorporation of meshes, biologic grafts, or biodegradable materials so that these larger structures maybe incorporated into a wound while tissue in-growth and wound healing occurs, which may result in faster closure. The pharmaceutical composition may be used to cover, close, wrap, pack, or fill open wounds. The pharmaceutical composition may be modified for each phase of wound healing as well as adapted to each individual's clinical problems and medical condition(s). The pharmaceutical composition may facilitate healing while simultaneously providing pain relief. This pharmaceutical composition may also be used in facilitating surgical wound closure, preventing wound infections, avoiding or decreasing keloid and hypertrophic scar formation, and preventing or decreasing inflammatory responses after implantation of vascular grafts, transplants, or prosthetic prosthesis. The pharmaceutical composition may also be used to treat grossly contaminated wounds, burn victims, and (chronic) venous stasis disease. The pharmaceutical composition may facilitate tissue and vessel in-growth.

In certain embodiments, the pharmaceutical composition may further include different components, characteristics, catalysts, or primers based on the phase of the healing process. The poloxamer may have different ingredients for adherence and tissue or matrix support depending on the phase of the wound.

To facilitate a better understanding of the present invention, the following examples of certain aspects of some embodiments are given. In no way should the following examples be read to limit, or define, the entire scope of the invention.

### EXAMPLES

### Example 1

As a specific example of the above, a biodegradable mesh, in a rolled gauze-like form, may be permeated in a poloxamer gel containing cefepime 2% and gentamicin 2%, with kaolin at a concentration of 5%, and a lidocaine concentration of 1%. Each of the components may be rendered sterile and may be placed in a Mylar pouch. The pouch may be opened and the biodegradable mesh roll covered in and permeated with poloxamer may be used to pack a wound. Any extra of the rolled biomaterial may be used as dressing helping to hold the packing in place. Extra poloxamer may squeezed out of the packet and used to finish filling the wound and wound margins, helping to adhere the substrate to the wound, as well as support wound healing, minimizing blood loss and pain in the process. Optionally, the pouch may further include a plastic backing which may be removed to expose an acrylate glue. The entire pouch may be affixed to cover the wound and may serve as an additional barrier against further infection.

### Example 2

Methods of preparing pharmaceutical compositions according to embodiments of the current disclosure are discussed below.

Granular or powdered poloxamer 407 may be irradiated or rendered sterile by chemical or other methods. A sufficient quantity of the poloxamer may then be hydrated in sterile water to obtain a final concentration of the poloxamer in the range of from 1% to 35% wt/volume. In particular the final poloxamer concentration may be 30% wt/volume. The resulting mixture of water and poloxamer may be kept at temperatures ranging from 2-10° C., until complete hydration takes place. This normally takes 24 hours for small volumes without any additional additives to the solution, and up to 48 hours for larger volumes, or if additives have been incorporated.

The mixture may be allowed to stay in the cold environment until completely hydrolyzed. Additional adjustments to the volume of the solution may take place, for example additional amounts of sterile water may be added periodically while the hydration process takes place until the final volume is reached.

In a particular embodiment, when sterility is a factor, the resulting hydrated gel may then bottled, sealed, and then autoclaved at a temperature of 121° C., 15 psi, for 15 minutes in a steam autoclave, or at 150° C. for 150 minutes in a dry-heat autoclave. The solution may then be allowed to cool to room temperature and then cooled to a temperature of about 2-10° C. for sterility and endotoxin testing. In other particular embodiments, sterility autoclaving may not be preformed.

In a particular embodiment, where a large amount of oxygen is desired to be incorporated into the gel, the gel while in the liquid phase may be taken into a Class 5 sterile environment. Using aseptic practices, a 0.22 micron filter may be attached mid line to a source of oxygen. From the filter, a sterile tube may be placed, with the end point placed into the solution. Filtered oxygen may then be allowed to bubble from the tube through the liquid solution. The top of the bottle may also be filled with pure oxygen and then capped. If desired, other chemical entities may be incorporated into the solution such as free-radical scavengers, oxidative or reductive species or classes of compounds, enzymes, or other entities that are affected or can be affected by oxygen and play a role in the functioning of tissue, the gel, the substrate, or any combination thereof. This embodiment may also be performed using aseptic techniques, or in a non-sterile fashion if sterility is not a concern.

In a particular embodiment, a sterilized 30% wt/volume poloxamer 407 solution may be supplemented with various factors that may help the substrate better perform its action, or may improve the local environment so that the substrate may maintain its integrity, may maintain the sterility of the location, or may improve the quality of the tissue matrix surrounding the pharmaceutical composition and the substrate. For example, in one application for attaching the composition to a site that has been colonized with MRSA, vancomycin may be reconstituted, filtered, and then added to the final product, so that the final concentration is 2%. This and other antibiotics, at concentrations ranging from 0.01% to 25%, or from 25%-99%, could be added for microbial suppression, biofilm degradation, prophylaxis, or any combination thereof.

In another embodiment, insulin, growth-factors, and tissue promoting factors may be added to the aqueous poloxamer solution, enabling both the adherence of human tissue to a burn site by the physical nature of the gel, and promoting the maintenance of the graft by said factors incorporated into the gel matrix. As a specific example, amastatin (a protease inhibitor) may be added along with epidermal growth factor (EGF), to the gel to promote tissue ingrowth when a human graft (the substrate) has been preformed and is being held in place by the poloxamer.

In certain embodiments, the sterilized poloxamers may be placed in sterile syringes or in packages for coating an application site. In one particular embodiment, the sterilized poloxamer may be dispensed in several 20 ml sterile syringes with an attached sterile tip. If the application site has an irregular shape, is in the sinus tracts, or presents other physiological difficulties for attachment, the gel may be refrigerated to convert it to its micelle solution form. After the application site has been cleaned and prepped, the poloxamer may then be placed into the site, coating it and the sides. The substrate may then be applied into the application site using aseptic techniques. The poloxamer may then hold the substrate to the wound.

### Example 3

Pharmaceutical compositions including poloxamers have been used to adhere collagen sheets, primarily Biopad® (Euroresearch, S.R.L., Milan, Italy), for assisting in the closure and healing of small and medium size (both acute and chronic) wounds. Using the pharmaceutical compositions including poloxamers, Biopad® (Euroresearch, S.R.L., Milan, Italy) has been successfully adhered to different depths of wound including partial thickness skin, subcutaneous tissue, and muscle wounds. The sizes of these wounds have varied from 1-2 cm up to almost 200 square centimeters. Various additives have been used in the pharmaceutical compositions, including vancomycin, gentamicin, zyvox, rifampin, nifedipine, dilantin, lidocaine, misprostasol, and aloe.

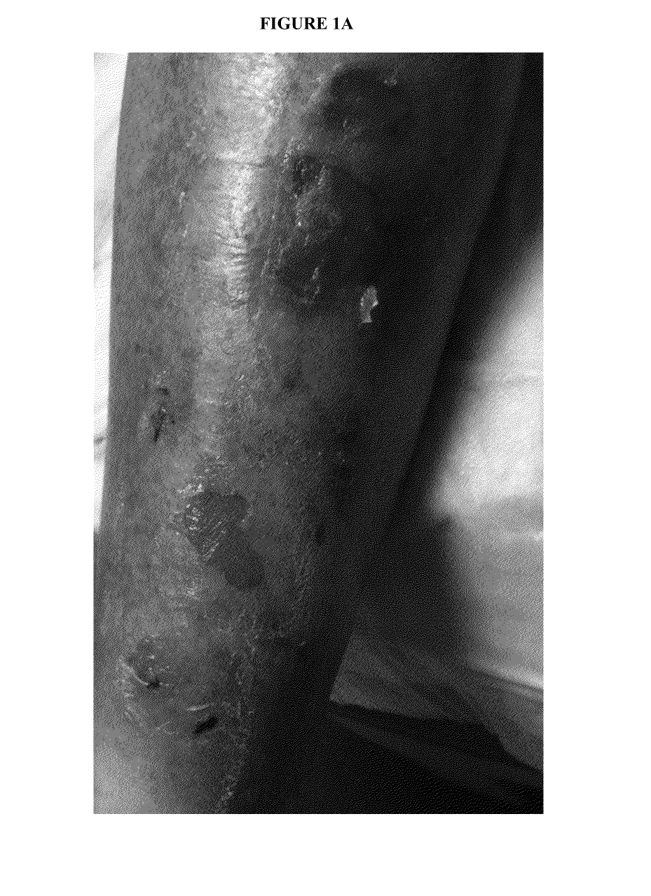

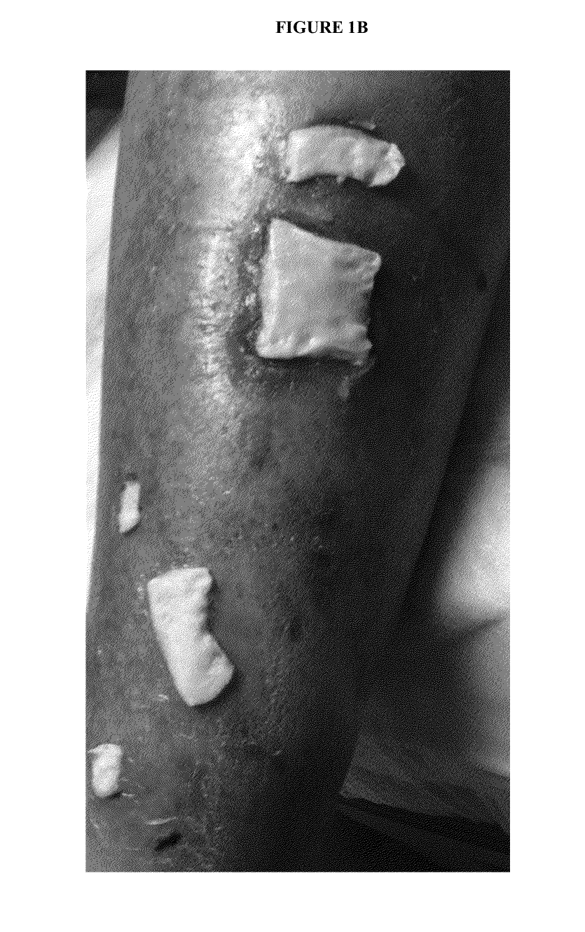

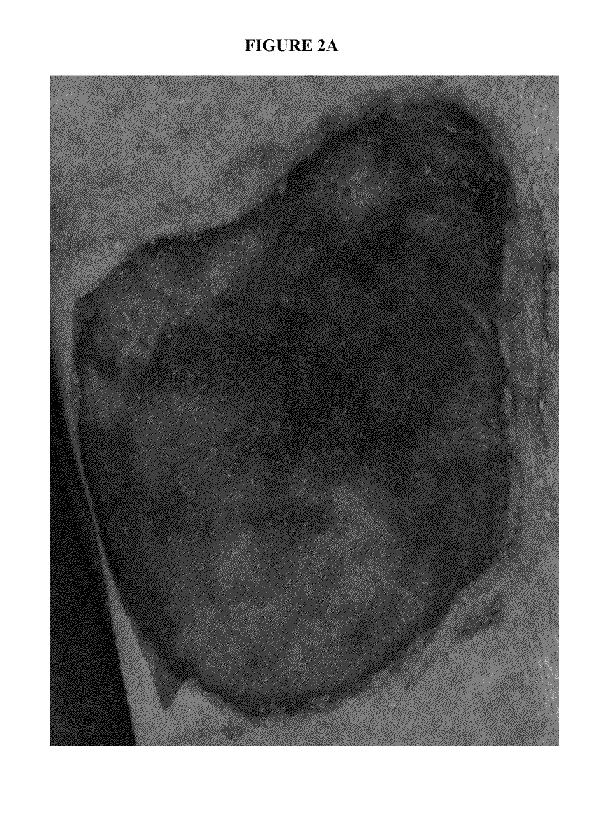

FIG. 1 shows a wound before treatment (A) and immediately after application of a pharmaceutical composition including a poloxamer and a substrate (B). The poloxamer composition used in this example comprised vancomycin 2%, gentamicin 2%, and poloxamer 407 30%. The substrate comprised Biopad®, a sponge shaped device, constituted exclusively by lyophilized type 1 native heterologous collagen extracted from horse flexor tendon.

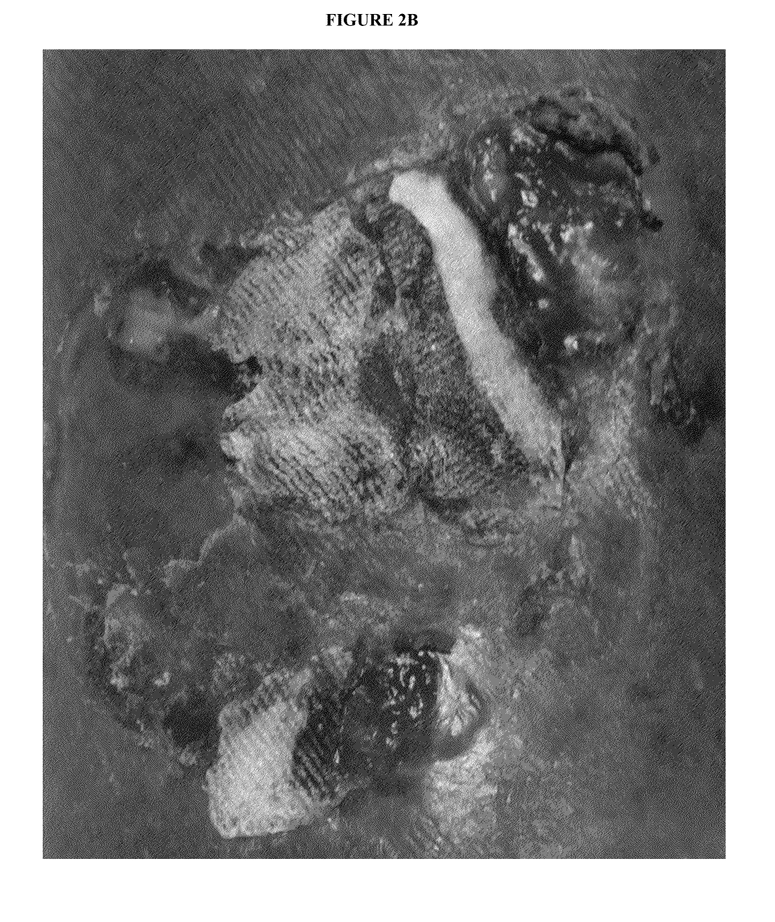

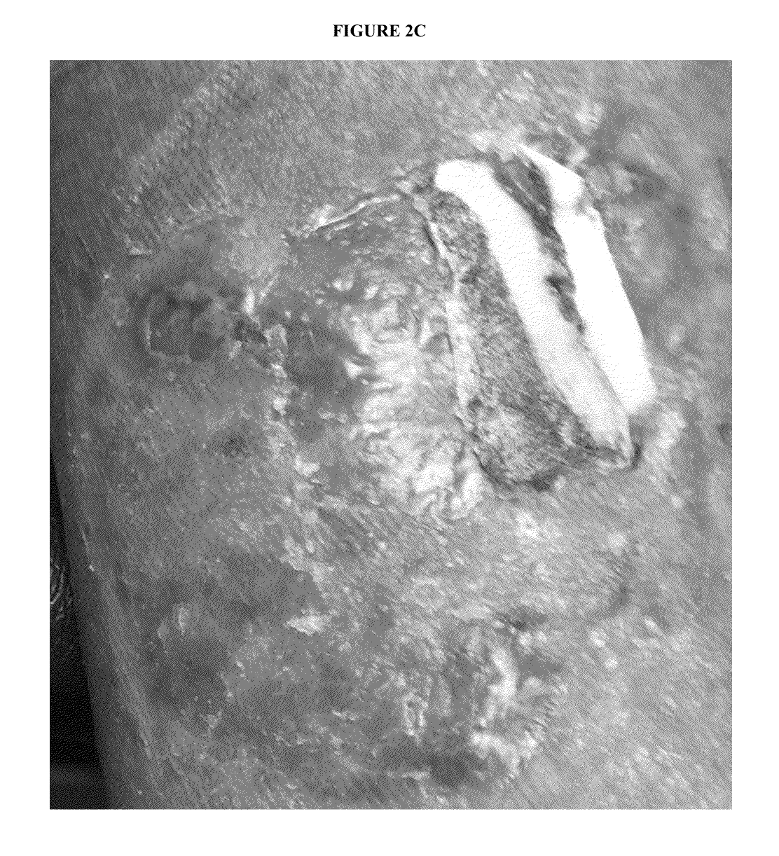

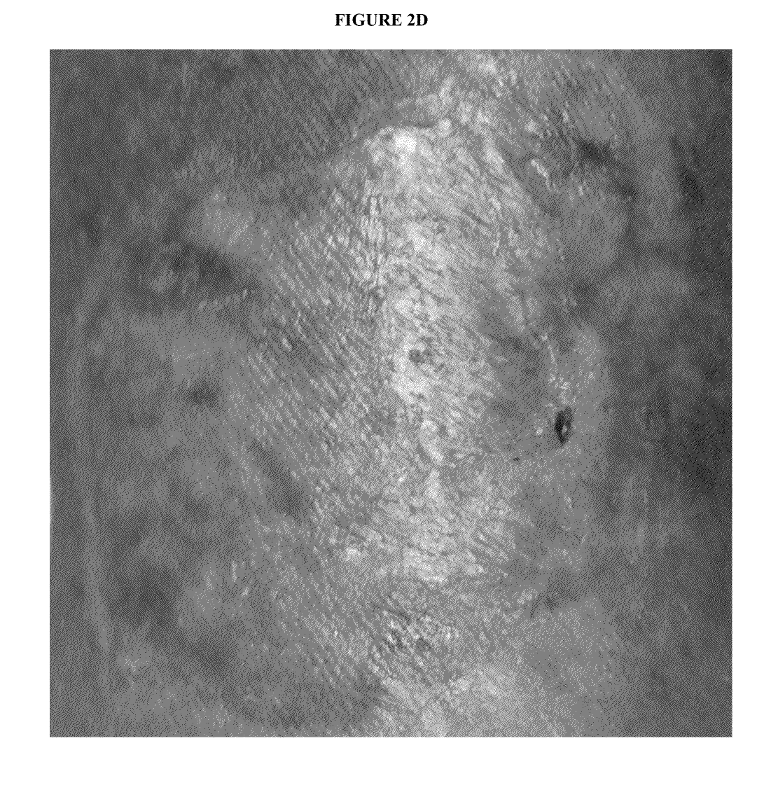

FIG. 2 shows another wound before treatment (A), 2 weeks after application of a pharmaceutical composition including a poloxamer and a substrate (B) (substrate can be seen adhering to the wound), another week after application of a pharmaceutical composition including a poloxamer and a substrate (C) (substrate can be seen adhering to the wound), and one month after application of a pharmaceutical composition including a poloxamer and a substrate (D), when wound closure is complete. The poloxamer composition comprises vancomycin 2%, gentamicin 2%, misoprostol 0.0024%, phenytoin 5%, and poloxamer 407 30%. The substrate comprised Biopad®. In addition, pharmaceutical compositions including poloxamers have been used to adhere dermis-derived tissue including Apligraf® (Organogenesis, Inc., Canton, Mass.), Dermagraft® (Advanced Biohealing, Inc., Westport, Conn.), TheraSkin® (Soluble Systems, Newport News, Va.), a biologically active cryopreserved real human skin allograft with both epidermis and dermis layers, and Allograft®. Pharmaceutical compositions including poloxamers have also been used on these type grafts later in the wound care process when other materials were initially applied. The poloxamer has been observed to facilitate healing (salvage) in such grafts after they have become grossly infected, as indicated by the observation of pus, or upon finding bacterial bio-film present in via positive culture. Late adherence of these dermis-derived grafts when they were not initially adherent (becoming incorporated) into these wounds has also been observed. These poloxamers have been used in conjunction with negative-pressure therapy with larger wounds successfully. Poloxamers have been used to fill tracts in tunneling wounds with success.

Poloxamers have been used with one autologous skin graft after initial application of the skin graft when areas of the skin graft had not achieved tissue in-growth or adherence. These areas of skin graft were essentially floating in the exudate from the wound on a patient with severe venous stasis disease in an irradiated skin cancer bed. Further successful incorporation (salvage) of areas of the non-adhered skin graft was observed. Not only was there further in-growth but more rapid skin bridging from surrounding native skin in areas that the skin graft failed was observed.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A method of attaching a substrate to a tissue comprising:
applying a substrate to a tissue; and
applying an aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, to the tissue in an amount sufficient for the poloxamer to hold the substrate to the tissue.

2. The method according to claim 1 further comprising impregnating the substrate with the aqueous solution.

3. The method according to claim 1, further comprising applying the aqueous solution at a temperature at which the poloxamer is in the form of micelles, than allowing the poloxamer to reach a temperature at which it forms hexagonal-packed cylinders.

4. A method of facilitating wound closure comprising:
applying an original substrate to a wound;
applying an original aqueous solution comprising about 1% to about 50% weight/volume poloxamer having the general formula HO(C₂H₄O)ₐ(C₃H₆O)_{b}(C₂H₄O)ₐH, wherein a ranges from 12-101 and b ranges from 20-56, to the wound in an amount sufficient for the poloxamer to hold the substrate to the wound; and
maintaining a substrate and poloxamer on the wound until wound closure.

5. The method according to claim 4 further comprising impregnating the substrate with the aqueous solution.

6. The method according to claim 4, further comprising applying the aqueous solution at a temperature at which the poloxamer is in the form of micelles, than allowing the poloxamer to reach a temperature at which it forms hexagonal-packed cylinders.

7. The method according to claim 4, further comprising:
removing the substrate and poloxamer from the wound; and
apply a new substrate and a new aqueous solution comprising a new poloxamer to the wound.

8. The method according to claim 7, wherein the new substrate and aqueous solution comprise the same materials as the original substrate and poloxamer.

9. The method according to claim 7, wherein the new substrate or poloxamer or both comprise different materials from the original substrate and poloxamer.

10. The method according to claim 7, wherein the substrate and poloxamer may be removed and a new substrate and aqueous solution may be applied more than twice prior to wound closure.

11. The method according to claim 7, wherein the new substrate and new poloxamer are selected based on a degree of healing of the wound.
